(19) United States
(12) Patent Application Publication
(10) Pub. No.: US 2002/0098472 A1
(43) Pub. Date: Jul. 25, 2002

(54) Method for inserting a microdevice or a nanodevice into a body fluid stream

(76) Inventors: Julian Van Erlach, Clifton Park, NY (US); Arlen L. Olsen, Clifton Park, NY (US); Jeffrey M. Smith, Pittsfield, MA (US); Laura B. Smith, Pittsfield, MA (US); Gerald E. Bender, Cheshire, MA (US); Audra L. Stinchcomb, Latham, NY (US); Denis P. Donnelly, Saratoga Springs, NY (US); Mark D. Scott, Clifton Park, NY (US); James E. Peterson, Delmar, NY (US); Robert S. Hirsch, Troy, NY (US)

Correspondence Address: ARLEN L. OLSEN, SCHMEISER, OLSEN & WATTS, 3 LEAR JET LANE, SUITE 201, LATHAM, NY, 12110, US

(21) Appl. No.: 09/727,718

(22) Filed: Nov. 30, 2000

(51) Int. Cl. C12Q 1/00; A61N 1/00

(52) U.S. Cl. 435/4; 607/1; 424/9.3

(57) ABSTRACT

A method for inserting a microdevice or nanodevice into a body fluid stream is disclosed. The micro or nanodevice may be inserted into or external to a cell, e.g., a red blood cell. For insertion into the cell, techniques such as reversible osmotic lysis, electroporation, microfine needle injection, and particle gun injection may be used. For the external device, it may be chemically modified with an organo hydroxyl, such as poly (ethylene glycol), methoxypoly (ethylene glycol). Also, it may include a lipid anchor.

### FIELD OF THE INVENTION

The present invention relates to micro and nanotechnology. In particular, the present invention relates to a method for inserting a microdevice or a nanodevice into a body fluid stream.

### BACKGROUND OF THE INVENTION

Heretofore, various methods and apparatus have been disclosed for using substrates in combination with biological members. U.S. Pat. No. 6,123,819 discloses an array of electrodes built on a single chip used to simultaneously detect, characterize and quantify individual proteins or biological molecules in solutions.

U.S. Pat. No. 6,051,380 discloses a microelectronic device designed to carry out and control complex molecular biological processes, including antibody/antigen reactions, nucleic acid hybridizations, DNA amplification, clinical diagnostics and biopolymer synthesis. None of the references, however, adequately describe attaching a substrate to a biological member for controlling and analyzing complex molecular biological processes and bodily conditions.

### SUMMARY OF THE INVENTION

In one aspect, the present invention includes a method of providing at least one of a microdevice, and a nanodevice; and inserting at least one of said microdevice and said nanodevice into a fluid stream within a body.

In a second aspect the present invention includes a method of providing at least one of a nanodevice and a microdevice; and inserting at least one of said nanodevice and said microdevice in a fluid stream within a body, wherein at least one of said nanodevice and said microdevice is extracellular.

### BRIEF DESCRIPTION OF THE DRAWINGS

FIG. 1 is a side view of a discoid human red blood cell and a 100 nm nanochip.

FIG. 2 is a circuit to measure temperature using a thermistor.

FIG. 3 illustrates the Electron Paramagnetic Resonance (EPR) spin probe detection method with an intracellular nanodevice.

FIG. 4 illustrates the nanotuning fork detection method with an intracellular nanodevice.

FIG. 5 illustrates the nanotuning fork detection method with an extracellular membrane bound nanodevice.

FIG. 6 illustrates the nanotuning fork detection method with a fluid phase nanodevice.

FIG. 7 illustrates the electron dense nanoprobe detection method with an intracellular nanodevice.

FIG. 8 is a side view of a discoid human red blood cell showing incorporation of the nanochip into the red blood cell via reversible osmotic lysis and resealing.

FIG. 9 is a side view of the star-shaped pore generated during osmotic lysis showing the lateral openings extending beyond the central pore.

FIG. 10 is a side view of a nanodevice anchored to a cell membrane via the attachment of a lipid tail.

FIG. 11 illustrates an extracellular nanodevice with methoxypoly(ethylene glycol) covalently linked to the nanodevice via substrate (nanochip) specific linker chemicals which utilize the free hydroxyl group of the polymer.

### DETAILED DESCRIPTION OF THE PREFERRED EMBODIMENTS

The present invention is based on advancements in the field of nanotechnology, which will allow monitoring, diagnosis, detection and modification of a biological member or a bodily function. The present invention may be used for controlling complex molecular biological processes such as antibody/antigen reactions, nucleic acid hybridizations, DNA amplification, clinical diagnostics, biopolymer synthesis and other cellular, subcellular and molecular activities by incorporating new instruments, machines and the like by attaching them to various human and animal cells or placing them within a biological fluid stream. In addition, the present invention may be used for detection, diagnosis, and monitoring of bodily conditions such as myocardial infarctions, stroke, sickle cell anemia, phlebitis and the like. Many other applications may also be readily apparent such as detection, diagnosis, and monitoring of mental, urinary, gastric, renal, vascular, lymphatic, uterine, endocrine (e.g., hormonal), drug levels and delivery, cancer, and the like.

In one embodiment, the present invention relates to a method and apparatus for attaching at least one of a microdevice and a nanodevice to a biological member. The apparatus can be attached to or implanted in a cell, tissue or organ. Additionally, the device may be external to a cell, tissue or organ (e.g., within a bodily fluid stream). The microdevice or nanodevice or apparatus is comprised of synthetic or synthetic and organic structures. Further, the apparatus may contain temperature, pressure, mechanical (e.g., harmonic) electrical, chemical and biological sensors and assays. In one embodiment, the apparatus may also contain a radio transmitter capable of transmitting continuous, interval, or on-demand readings from a monitor. The transmitter will contain a power supply, such as a battery. In another embodiment, both the transmitter and power supply will be incorporated on a single chip. The apparatus may contain remotely programmable units, capable of manipulating, detecting and analyzing temperature, pH, blood cell count, pressure, electrical, chemical (such as iron deficiency) and biological sensors according to time and location. For example, oxidant stress may be detected for treatment of acute anemia, stroke, myocardial infarction and the like.

The biological member may include either a human or animal cell, organ, or tissue. Further, the biological member may include one or more of a blood cell, a lipid molecule, a liver cell, a nerve cell, a skin cell, a bone cell, a lymph cell, an endocrine cell, a circulatory cell, a muscle cell or the like.

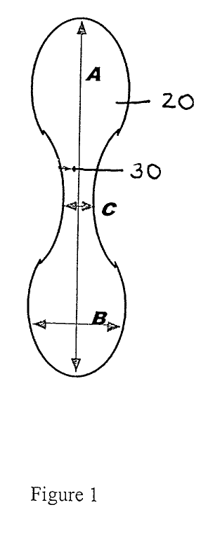

Referring now to FIG. 1, a nanodevice or microdevice 30 may be operatively attached to red blood cell 20 in one embodiment. The normal, mature discoid human red blood cell 20 has a mean diameter A of approximately 8 μm, a mean cell thickness B (comprising rim and central thickness) of approximately 1.7 μm, a single cell volume of approximately 95 fl, and a surface area of approximately 135 sq. μm. Typical capillary sizes are approximately 3-4 μm and typical splenic sinusoids are approximately 1 μm. Therefore, a microdevice or nanodevice of 100 nm may be accommodated within the volume of a normal human red blood cell 20 (mean diameter of approximately 8 μm or the red blood cells of other animal species with a mean diameter of approximately 5-10 μm). Intracellular inclusion of the nanodevice or microdevice 30 should not adversely affect red blood cell structure or function, but will vastly extend the circulation time of the nanochip. For example, human red blood cells circulate for 120 days while murine (mouse) cells survive for 50 days. In contrast, unmodified extracellular nanodevices or microdevices free within the blood stream would likely have survival times of minutes to hours due to mechanisms such as phagocytosis or other immunological reactions.

In one embodiment, the nanodevice or microdevice of the present invention includes a semiconductor surface, formed of material substrates such as semiconductor materials as gallium arsenide, silicon or silicon oxide. Further, scanning tunneling microscopy, which produces nanodevices (only a few Angstroms in diameter, or a single or few atomic layers thick) can be used to manipulate single atoms on the semiconductor surface. These nanodevices, which serve as molecular electrodes, are built using various chemical techniques. The electrode may have differing electrochemical properties which can be made to correspond with numerous biological molecules, including biochemicals and proteins. For example, one method of constructing circuit features of less than 300 Angstroms is disclosed in U.S. Pat. No. 6,049,131 and assigned to International Business Machines Corporation. The '131 patent discloses forming NFET and PFET (Field Effect Transistors) structures using a method of selective refractory metal growth/deposition on exposed silicon, but not on the field oxide.

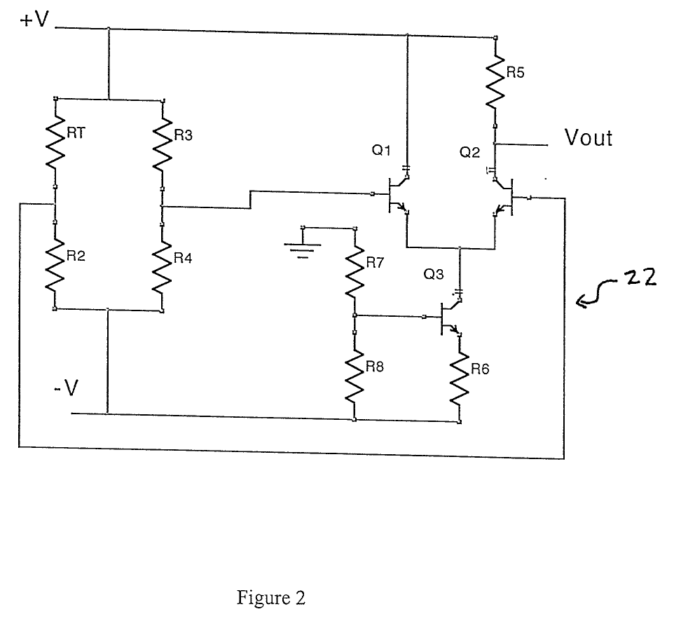

Referring now to FIG. 2, the nanodevice or microdevice of the present invention incorporates at least one circuit feature thereon, generally 22. The circuit feature may include a diagnostic system, a transmitter, a receiver, a battery, a transistor (Q1, Q2 and Q3 in FIG. 2), a capacitor and a detector. For example, nanoelectrode arrays may be used to detect, characterize and quantify single molecules in solution such as individual proteins, complex protein mixtures, DNA and other molecules in vivo. Such nanoelectode arrays are disclosed in U.S. Pat. No. 6,123,819 and assigned to Protiveris, Inc.

In an embodiment, the apparatus contains an active or passive location and data transmission method. The apparatus may contain an on-board power source, such as a battery, radio transmitter and receiver, laser and a programmable logic unit.

In another embodiment, the apparatus may include an active or passive tag or detector that is attached to a particular cell, including a red blood cell. Each tag is identified by a unique code. The active tag may include a transmitter that transmits the unique code and the passive tag includes an element that vibrates and interacts with signals sent from a plurality of detector systems. The detector system includes both a transmitter and a receiver. These tags act as a tracking system to identify the movement of a specific cell in the body. In one aspect, the transmitter sends a signal to the passive tag element and the element responds. In another aspect, the receiver of the present apparatus, determines the unique code of the element and a processing system receives the information from at least two detector systems. A triangulation method is used to determine the location of the tag.

Nanodevices exist today in very basic implementations only. Nanodevices are built in two distinct ways, top down using lithographic/chemical processes or bottom up (molecule by molecule) using chemical synthesis/atomic force microscope techniques. Both techniques allow development of features and devices in the 1-100 mn size range. Most devices created so far have been in university or research laboratory type settings and are not available in commercial quantities.

Nano size particles are available commercially and can be used as a first step in passive biological sensor applications. Companies like Nanoprobes, Inc. of Yaphank, N.Y. and Vector Labs of Burlingame, Calif. commercially sell nano size particles in a variety of materials including electron dense materials such as gold.

Resonance type nanodevices also exist. Caltech has demonstrated a 10×10×100 nm resonant GaAs beam. This device resonates at 7 GHz when a voltage is applied at the base to excite the beam. Cornell University has created a resonant Harp that has strings that are 50 nm in diameter and 1 to 8 microns long. Again, an applied voltage is used to create resonance in the structure. Georgia Tech has used carbon tubes as vibrating beams, exciting the natural frequency of the structure by applying a modulated current to the base of the structure. They have demonstrated that the mass of an object attached to the end of the tube can be calculated by the change in the resonant frequency of the structure. Spring constants of single polymer chains have also been measured for chains of polystyrene. If a nano size modulated power source could be used to excite these nanodevices in vivo, detection of resonance and resonance changes of these nanodevices would be easily accomplished using magnetic resonance technologies. However, no power source on this scale is available. Therefore other technologies must be utilized for an in vivo approach.

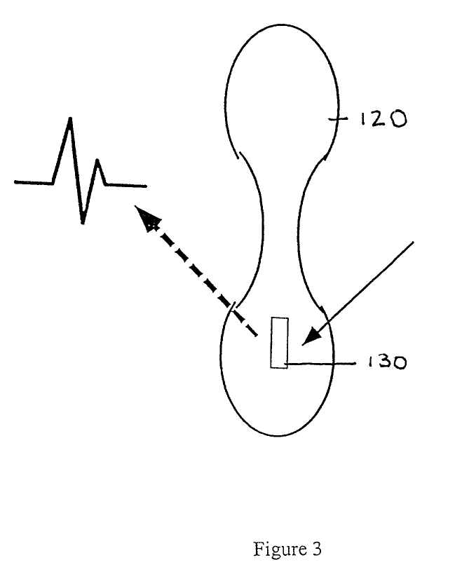

A technology that is applicable for nanodevice sensory detection is Electron Spin Resonance (ESR) or Electron Paramagnetic Resonance (EPR). Referring now to FIG. 3, EPR 24 is the process of resonant absorption of microwave radiation by paramagnetic ions or molecules, with at least one unpaired electron spin, and in the presence of a static magnetic field. EPR can be used to detect free radicals, odd electron molecules, transition metal complexes, lanthanade ions, and triplet state molecules in vivo. Some examples of detectable materials include phosphorus, arsenic, sulphur, germanium, and organic free radicals such as Di-phenyl-b-picryl-hydrazyl (DPPH). Detectable spin probes based on nitroxide free radicals can be used to detect biological activity such as oxidant stress and pH levels. Concentrations of spin probes can be used to enhance the sensitivity of EPR technology.

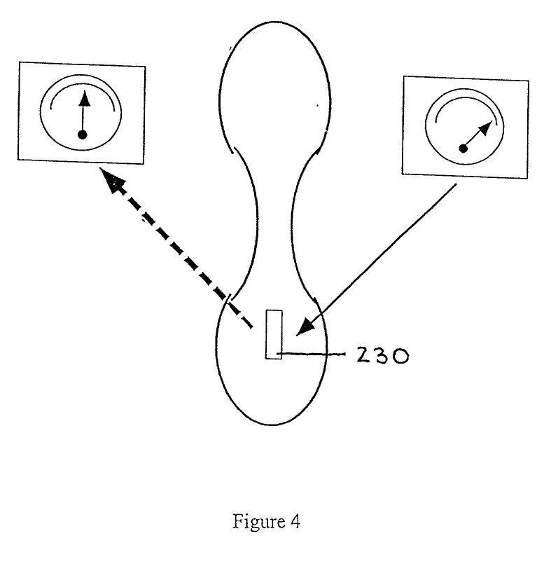

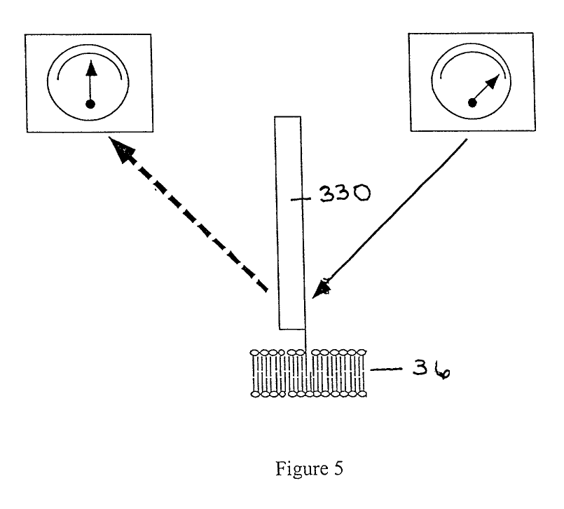

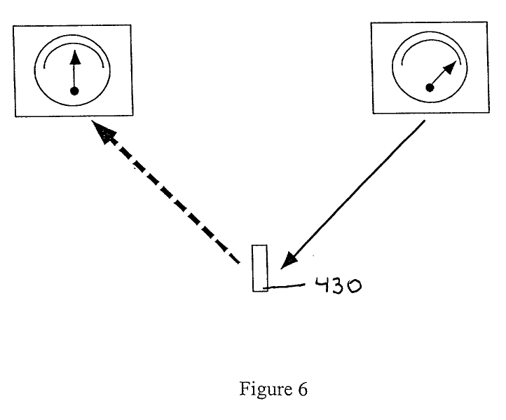

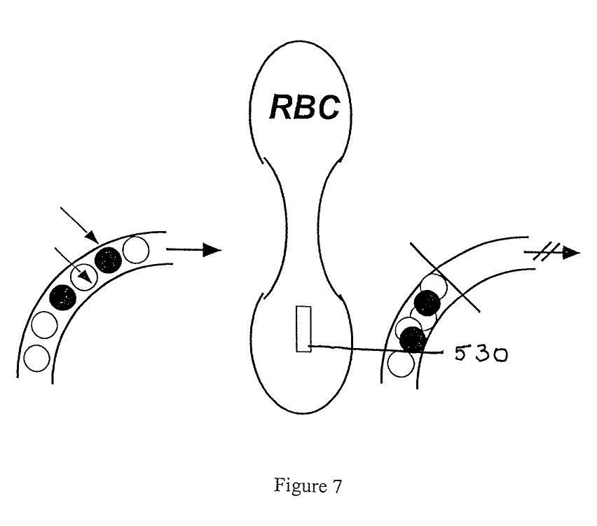

Referring now to FIGS. 4-6, another technology applicable for nanodevice sensory detection is a nanotuning fork detection method. FIG. 4 illustrates the nanotuning fork detection method with an intracellular nanodevice 230. FIG. 5 illustrates the nanotuning fork detection method with an extracellular membrane 36 bound nanodevice 330. FIG. 6 illustrates the nanotuning fork detection method with a fluid phase nanodevice 430. The nanotuning fork can be either unmodified or modified with poly(ethylene glycol) or its derivatives. Referring now to FIG. 7, electron dense nanoparticles or nanodevices 530 with spin probes attached can be used as passive blood flow sensors for determining pathologic changes in tissue blood flow. These nanodevices can be used for in vivo blood flow detection utilizing Nuclear Magnetic Resonance (NMR) technologies. These nanodevices will allow the measurement of blood flow and the detection of any blockages that may inhibit the flow of blood.

NMR technology places a substance in a strong magnetic field that affects the spin of the atomic nuclei of certain isotopes of common elements. Radio wave frequencies passes through the substance then reorients these nuclei. When the wave is turned off, the nuclei release a pulse of energy that provides data on the molecular structure of the substance and that can be transformed into an image by computer techniques. Typical substances that can be used for NMR spectroscopy and imaging are shown in Table 1.¹ 1.ⁱ

TABLE 1
Typical NMR Substances

| Nuclei | Unpaired Protons | Unpaired Neutrons | Net Spin | γ(MHz/T) |
|---|---|---|---|---|
| ¹H | 1 | 0 | ½ | 42.58 |
| ²H | 1 | 1 | 1 | 6.54 |
| ³¹P | 0 | 1 | ½ | 17.25 |
| ²³Na | 2 | 1 | 3/2 | 11.27 |
| ¹⁴N | 1 | 1 | 1 | 3.08 |
| ¹³C | 0 | 1 | ½ | 10.71 |
| ¹⁹F | 0 | 1 | ½ | 40.08 |

In another embodiment, the present apparatus includes an active or passive tag or detector attached to a microdevice or nanodevice. Hereinafter, “micromachine” refers to both a “micromachine” and a “nanomachine”. Machines outside of the body can be used to control this micromachine. In one aspect, this micromachine can be used to perform surgery. In another aspect the micromachine is used for analysis. In still another aspect, this micromachine can be used to deliver drugs to selected cells in the body.

Further, in another embodiment, the apparatus may contain a navigation system, propulsion system (hydraulic, chemical, turbine, electrical, mechanical, or other), methods for attachment to tissue (anchors and legs), molecular assays (bio-reactants) for testing presence of proteins and other compounds and drug, chemical, and radiation delivery means. In one embodiment, all of these would be incorporated on a single chip.

Fabrication methods used to produce the powerful, integrated circuits may include electron beam lithography, ion beam lithography, x-ray lithography, spatial phase-locked lithography and molecular beam epitaxy. Electron beam lithography exposes a pattern directly on the wafer using an electron beam. Materials used in electron beam lithography may include gold, titanium, silver, sapphire and polyimide.

Various chemical processes for pattern transfer during electron beam lithography may include electroplating or dry etching. Dry etching has the capability of producing structures in the range of approximately 10 nm. A solid state substrate is etched via ion bombardment (plasma etch) or chemical reaction (chemical etch) in a specified gaseous environment vs. a liquid environment.

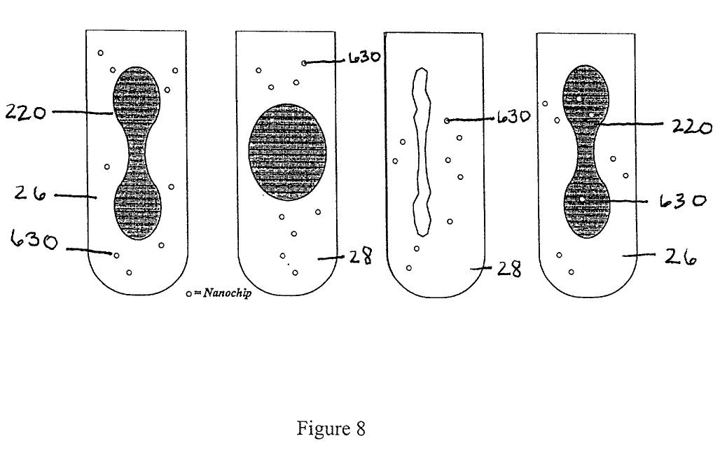

In one embodiment, incorporation of a nanodevice or microdevice inside the biological member can be done via reversible osmotic lysis. Referring now to FIG. 8, this procedure has been used to incorporate both small and large proteins, including hemoglobins (Tetramers of 64,000 Da) with a mean diameter of approximately 5.5 nm; and catalase (Tetramer of approximately 264,000 Da) with a mean diameter of approximately 15 nm, as well as large, linear, dextran molecules of molecular weights of approximately 500,000 Da. Some efficacy of entrapment is even noted with 2,000,000 Da dextrans, but with vastly decreasing efficacy. Further, larger particles, up to 100 nm, are incorporated osmotically into resealed red blood cells. When a red blood cell 220 is placed in an isotonic solution 26, said red blood cell 220 maintains a normal discoid shape. However, placing said red blood cell 220 in a hypotonic solution 28 produces cellular swelling and lysis. Cellular lysis of the red blood cell 220 results in the collapse and extrusion of intracellular constituents and a mixing with extracelluar nanochips 630. Upon restoration of isotonicity, the red blood cell 220 cytoskeleton returns to the normal discoid shape pulling extracellular proteins and nanochips 630 into said red blood cell. Previous studies indicate that approximately 30% of the exogenous agent is incorporated into the resealed red blood cell.

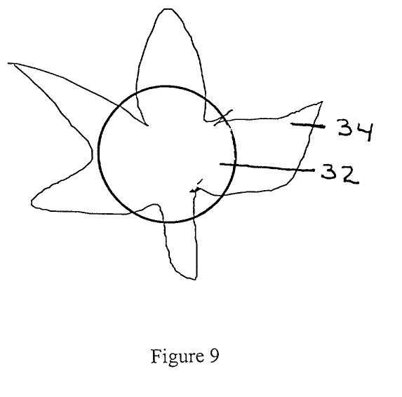

Referring now to FIG. 9, the pore diameter generated during osmotic lysis is typically approximately 50 nm, though some extreme estimates exist of pores up to 1000 nm in diameter. The heterogeneity of the reported pore diameter likely results from the physical nature of this transient pore. In one embodiment, the pore exists in a star shaped configuration with a stable central channel 32 (approximately 50 nm) with less stable side channels 34 extending laterally beyond the central pore. This hypothesis is supported by studies with large linear polymers which demonstrate that the incorporation of larger compounds (approximately 100 nm) can be accomplished. Based on these studies, as well as other experimental evidence, particles of ≦50 nm are very efficiently incorporated into osmotically resealed red blood cells. Particles in the 500-100 nm range are incorporated, but with decreasing efficacy.

For tissues not amenable to osmotic lysis and resealing (e.g., nucleated cells), nanodevices can be intracellularly introduced via several different methods.

One technique is virtually identical to that currently used in cloning technology in which a microfine needle is used to inject small fluid amounts directly into the cells cytoplasm or nucleus. The injected fluid contains one or more nanodevices.

A second method is via direct particle gun injection; a technique analogous to a gun firing a bullet. An example of this technology is the use of gold beads (in the nm to low μm range) to which DNA is attached. The Gold bead is shot out of a particle gun with a defined force to penetrate the cell membrane and/or nuclear membrane depositing the bead within the cell.

A third means of incorporating nanodevices intracellularly is via electroporation. In this method an electrical current is passed through the media containing the cells of interest. The electrical current is used to create membrane pores that allows the diffusion or active driving of the nanodevice into the cytoplasm of the cells. This technique is usable on all cells and can also be used on tissues. When using the appropriate protocols this method does not affect the cellular viability.

The word “electroporation” is used to describe the use of a transmembrane electric field pulse to induce microscopic pores in a membrane. These pores are commonly called “electropores.” Their presence allows molecules, ions, and water to pass from one side of the membrane to the other. Electropores are located primarily on the surfaces of cells which are closest to the electrodes. If the electric field pulse has the proper parameters, then the “electroporated” cells can recover (the electropores reseal spontaneously) and cells will continue to grow and express their genetic material. Throughout the 1980s the use of electroporation became very popular because it was found to be an exceptionally practical way to place drugs, genetic material (e.g., DNA), or other molecules into cells. Since the late 1980s, scientists began to use electroporation protocols for molecular delivery applications on multicellular tissue.

The upper limit current threshold determines sensitive and resistant cells. Cell toxicity occurs when pore diameter and total pore area become too large for the cell to repair by any spontaneous or biological process. This causes the cell to be irreversibly damaged. To prevent this damage, pulse protocols are empirically developed for the tissue in question.

Although early research on electropore mediated transport across membranes assumed that simple thermal motion (i.e. diffusion) propelled molecules through electropores, research in the late 1980s and early 1990s began to reveal that movement of molecules through electropores depends on other experimental conditions and pulse electrical parameters in a way that indicates that additional poorly understood processes are involved. These reports show that certain experimental conditions and parameters of electrical pulses may be capable of causing many more molecules to move per unit time than simple diffusion. For example, there is good evidence that molecular flow is influenced by molecular charge and current. This implies a polarity dependence in electroporation. Although this apparent contradiction will have to be resolved by future basic research, it clearly suggests that pulsers with more adjustable electrical parameters will be advantageous in protocol development.

An additional important consideration in all electroporation protocols is that during the pulse the electric field causes electrical current to flow through the cell suspension or tissue. Biologically-relevant buffers for cells, and bathing media as well as fluid in extracellular space in tissues contain ionic species at concentrations high enough to cause high electric currents to flow. Electrical parameters of porating pulses can be used which could lead to dramatic and biologically unacceptable heating and other unwanted effects to take place. One way to avoid or minimize the heating is to use a relatively high amplitude, short duration square wave pulse instead of an exponentially-decaying pulse. Principles of physics and studies of electroporation mechanisms suggest that the early part of exponentially decaying pulses does most of the membrane porating but the later part only continues to heat the medium. A second strategy is to use two short duration pulses instead of one pulse with a duration equal to the sum of the two short pulses.

There have been two main waveform categories of porating pulses. They are: i) exponentially-decaying, and ii) square wave pulses. These waveform qualities were dictated by principles of electrical engineering and the fact that pulsers designed for one waveform usually could not deliver the other waveform. In cases where there is evidence that an exponentially decaying pulse may have an advantage for a particular application, a protocol which delivers two pulses, one which is high in amplitude and short in duration followed by a second which is low in amplitude but long in duration, may simulate the effects of the exponentially-decaying pulse or even provide an improved result.

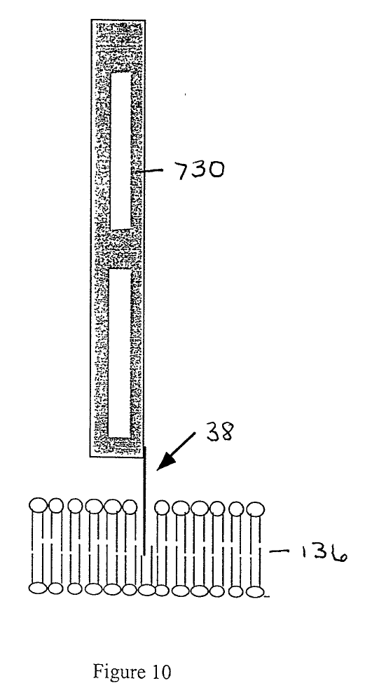

Referring now to FIG. 10, a nanodevice or microdevice 730 may also be anchored to a cell membrane 136 via the attachment of a lipid tail or phospholipid anchor 38. This device is suitable for all tissues and can be accomplished by simply applying (e.g., via a drop of liquid) the device to the tissue of interest.

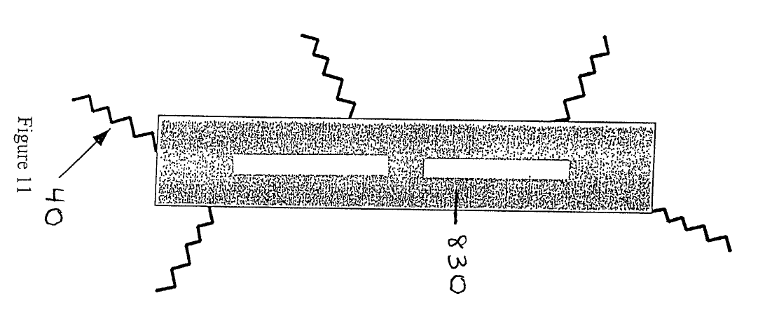

Further embodiments include solid tissue phase applications such as extracellular tissue implants, either unmodified or biomaterial modified, e.g., poly(ethylene glycol) modified. Referring now to FIG. 11, extracelluar nanodevices or microdevices 830 can be either unmodified or chemically modified to prolong vascular (or other fluid) retention, prevent immunologic detection (e.g., phagocytosis), or unwanted endocytosis by cells. Nanochip modification is initially envisioned using poly(ethylene glycol) [two free terminal hydroxyl groups] or methoxypoly(ethylene glycol) [one free terminal hydroxyl group]. Poly(ethylene glycol) and its derivatives are nonimmunogenic polymers which enhance vascular retention and prevent/diminish phagocytosis, endocytosis, or immune complex-mediated clearance. Methoxypoly(ethylene glycol) 40 can be covalently linked to the nanodevice via substrate (nanochip) specific linker chemicals which utilize the free hydroxyl group of the polymer.

In one embodiment, the apparatus can be ingested or injected into a cell, circulatory, lymph, cerebrospinal or digestive system. In one embodiment, said apparatus is free-floating. The device may free float or target a specific location within the body in order to perform a designated function for which it has been specifically equipped. For example, in one embodiment, the apparatus and method could be used to deliver drugs to a target area of the body, including specific cells.

Potential applications of first-generation nanodevices are numerous. Nanodevices can be used for enhanced visualization of vascular occlusion (partial or complete) as well as providing intravascular (e.g., capillary) red blood cell velocity via nanotuning fork devices or electron dense nanodevices. This is useful in the detection of, e.g., myocardial infarctions, stroke, sickle cell anemia (both stroke and painful crisis) and phlebitis (deep vein clotting). Further, nanodevices can be used in the detection of vascular aneurysms. Pooling of nanodevices aid in diagnosing and localizing the site of the aneurysm. Use of membrane bound and/or intraerythorcyte (red blood cell) nanodevices coupled to spin probes would allow measurement of oxidant stress at either the whole body or organ level. Oxidant stress is an indication of acute anemia, stroke, myocardial infarction, etc.

The nanodevices can aid in the generation of pH sensitive electrical circuits for determination of body fluid (e.g., blood, urine, cerebral spinal fluid, lymph). This application would be of potential diagnostic benefit in ischemia-reperfusion injury, kidney disease, and central nervous system injury.

Nanodevices can also be used as indicators of specific biologic activity. Ferrous nanodevices coupled with changeable indicators of specific intracellular biologic activity would allow cellular constituents to be separated for simplified analysis of in vivo enzyme activation.

We claim:

1. A method comprising
providing at least one of a microdevice and a nanodevice; and
inserting at least one of said microdevice and said nanodevice into a fluid stream within a body.

2. The method of claim 1, further comprising the step of inserting at least one of said microdevice and said nanodevice into a cell.

3. The method of claim 2, wherein said cell is a red blood cell.

4. The method of claim 1, wherein the step of inserting further comprises the step of inserting the substrate into said cell via at least one of reversible osmotic lysis, electroporation, microfine needle injection, and particle gun injection.

5. The method of claim 1, wherein said biological member is selected from the group consisting of a blood cell, lipid molecules, a liver cell, a nerve cell, a skin cell, a bone cell, a lymph cell, an endocrine cell, a circulatory cell, and a muscle cell.

6. The method of claim 1, wherein at least one of said nanodevice and said microdevice is selected from the group consisting of a diagnostic system, a transmitter, a receiver, a battery, a transistor, a capacitor, and a detector.

7. The method of claim 1, wherein at least one of said nanodevice and said microdevice is inserted within said biological member.

8. The method of claim 1, wherein said biological member is one of a red blood cell and lipid molecules.

9. The method of claim 1, wherein at least one of said nanodevice and said microdevice has a substrate selected from the group consisting of Gallium Arsenide, silicon, and silicon oxides.

10. The method of claim 1, wherein at least one of said nanodevice and said microdevice is formed using one of optical lithography, electron beam lithography, ion beam lithography, X-ray lithography, and spatial phase-locked electron beam lithography.

11. The method of claim 1, wherein at least one of said nanodevice and said microdevice is a resonance type nanodevice.

12. The method of claim 1, wherein at least one of said nanodevice and said microdevice is adapted for detection by one of electron paramagnetic resonance (EPR), electron spin resonance (ESR) and nuclear magnetic resonance (NMR).

13. The method of claim 1, wherein EPR detects molecules selected from the group consisting of free radicals, odd electron molecules, transition metal complexes, lanthanade ions and triplet state molecules.

14. The method of claim 1, wherein at least one of said nanodevice and said microdevice includes a material selected from the group consisting of phosphorus, arsenic, sulfur, germanium and organic free radicals.

15. A method comprising:
providing at least one of a nanodevice and a microdevice; and
inserting at least one of said nanodevice and said microdevice in a fluid stream within a body, wherein at least one of said nanodevice and said microdevice is extracellular.

16. The method of claim 15, further comprising the step of chemically modifying at least one of said nanodevice and said microdevice such that it is adapted to prolong vascular retention, prevent immunologic detection, or prevent unwanted endocytosis by cells.

17. The method of claim 15, further comprising the step of chemically modifying at least one of said nanodevice and said microdevice with an organo hydroxyl.

18. The method of claim 17, wherein said organo hydroxyl group is selected from the group consisting of poly (ethylene glycol), methoxypoly (ethylene glycol).

19. The method of claim 15, further comprising attaching a lipid anchor to at least one of said nanodevice and said microdevice with an organo hydroxyl.
